SEQUENCE LISTING (6 sequences)

SEQ ID NO: 1 - DNA, 2847 bp, Rattus norvegicus
cagctccaaggcacttgctccatttggggtgtgcctgcacctagctggttgcaaattgggccacagaggatctggaaaggatggaacaacgggctagcttagactcagaggagtctgagtccccaccccaagagaactcctgcctggaccctccagacagagaccctaactgcaagccacctccagtcaagccccacatcttcactaccaggagtcgtacccggctttttgggaagggtgactcggaggaggcctctcccctggactgcccttatgaggaaggcgggctggcttcctgccctatcatcactgtcagctctgttctaactatccagaggcctggggatggacctgccagtgtcaggccgtcatcccaggactccgtctccgctggtgagaagcccccgaggctctatgatcgcaggagcatcttcgatgctgtggctcagagtaactgccaggagctggagagcctgctgcccttcctgcagaggagcaagaagcgcctgactgacagcgagttcaaagacccagagacaggaaagacctgtctgctaaaagccatgctcaatctgcacaatgggcagaatgacaccatcgctctgctcctggacgttgcccggaagacagacagcctgaagcagtttgtcaatgccagctacacagacagctactacaagggccagacagcactgcacattgccattgaacggcggaacatgacgctggtgaccctcttggtggagaatggagcagatgtccaggctgcggctaacggggacttcttcaagaaaaccaaagggaggcctggcttctactttggtgagctgcccctgtccctggctgcgtgcaccaaccagctggccattgtgaagttcctgctgcagaactcctggcagcctgcagacatcagcgcccgggactcagtgggcaacacggtgcttcatgccctggtggaggtggcagataacacagttgacaacaccaagttcgtgacaagcatgtacaacgagatcttgatcctgggggccaaactccaccccacgctgaagctggaagagatcaccaacaggaaggggctcacgccactggctctggctgctagcagtgggaagatcggggtcttggcctacattctccagagggagatccatgaacccgagtgccgacacctatccaggaagttcaccgaatgggcctatgggccagtgcactcctccctttatgacctgtcctgcattgacacctgtgaaaagaactcggttctggaggtgatcgcttacagcagcagtgagacccctaaccgtcatgacatgcttctcgtggaacccttgaaccgactcctacaggacaagtgggacagatttgtcaagcgcatcttctacttcaacttcttcgtctactgcttgtatatgatcatcttcaccgcggctgcctactatcggcctgtggaaggcttgcccccctataagctgaaaaacaccgttggggactatttccgagtcaccggagagatcttgtctgtgtcaggaggagtctacttcttcttccgagggattcaatatttcctgcagaggcgaccatccctcaagagtttgtttgtggacagctacagtgagatacttttctttgtacagtcgctgttcatgctggtgtctgtggtactgtacttcagccaacgcaaggagtatgtggcttccatggtgttctccctggccatgggctggaccaacatgctctactatacccgaggattccagcagatgggcatctatgctgtcatgattgagaagatgatcctcagagacctgtgccggtttatgttcgtctacctcgtgttcttgtttggattttccacagctgtggtgacactgattgaggatgggaagaataactctctgcctatggagtccacaccacacaagtgccgggggtctgcctgcaagccaggtaactcttacaacagcctgtattccacatgtctggagctgttcaagttcaccatcggcatgggcgacctggagttcactgagaactacgacttcaaggctgtcttcatcatcctgttactggcctatgtgattctcacctacatccttctgctcaacatgctcattgctctcatgggtgagaccgtcaacaagattgcacaagagagcaagaacatctggaagctgcagagagccatcaccatcctggatacagagaagagcttcctgaagtgcatgaggaaggccttccgctctggcaagctgctgcaggtggggttcactcctgacggcaaggatgactaccggtggtgtttcagggtggacgaggtaaactggactacctggaacaccaatgtgggtatcatcaacgaggacccaggcaactgtgagggcgtcaagcgcaccctgagcttctccctgaggtcaggccgagtttcagggagaaactggaagaactttgccctggttccccttctgagggatgcaagcactcgagatagacatgccacccagcaggaagaagttcaactgaagcattatacgggatcccttaagccagaggatgctgaggttttcaaggattccatggtcccaggggagaaataatggacactatgcagggatcaatgcggggtctttgggtggtctgcttagggaaccagcagggttgacgttatctgggtccactctgtgcctgcctaggcacattcctaggacttcggcgggcctgctgtgggaactgggaggtgtgtgggaattgagatgtgtatccaaccatgatctccaaacatttggctttcaactctttatggactttattaaacagagtgaatggcaaatctctacttggacacat

SEQ ID NO: 2 - DNA, 2768 bp, Rattus norvegicus
ctgctctgtccactgtgtgagacgaacaggtggagggtggacgacgcagagaaagctcggagcgggccgcggaggttcccacagccccattactgtcagcgttgagccgcacccctccgggccgcacttcctctctcagtccccgctgccggagagccccgctaggctcggtgatcctagcctgcagtttgccgccgctacaccttggcttcagcctgcgggcccctctccatcaccttctccaggtcccagccaggcctgcccctgcggtatgagagaggaaccttaacatctccatctctacagaggtttcagctgtaaggagcatcctcctctctcaggatgacttcagcctccagccccccagctttcaggctggagacttccgatggagatgaagagggcaatgctgaggtgaacaaggggaagcaggaaccgccccccatggagtcaccattccagagggaggaccggaattcctcccctcagatcaaagtgaacctcaacttcataaagagacctcctaaaaacacttctgctcccagccagcaggagccagatcggtttgaccgtgaccgactcttcagtgtggtctcccggggtgtccccgaggaactgactggactgctagaatacctgcgctggaacagcaagtacctcactgactctgcatacacagaaggctccactggaaagacgtgcctgatgaaggctgtgctgaaccttcaggatggggtcaatgcctgcatcatgccgctgctgcagattgacaaggattccggcaatcccaagcccctcgtcaatgcccagtgcatcgatgagttctaccaaggccacagtgcgctgcacatcgccatagagaagaggagcctgcagtgcgtgaagctgctggtagagaatggagcggatgttcacctccgagcctgtggccgcttcttccaaaagcaccaaggaacttgtttctattttggagagctacctctttctctggctgcgtgcaccaagcagtgggatgtggtgacctacctcctggagaacccacaccagccggccagcctggaggccaccgactccctgggcaacacagtcctgcatgctctggtaatgattgcagataactcgcctgagaacagtgccctggtgatccacatgtacgacgggcttctacaaatgggggcgcgcctctgccccactgtgcagcttgaggaaatctccaaccaccaaggcctcacacccctgaaactagccgccaaggaaggcaaaatcgagattttcaggcacattctgcagcgggaattctcaggaccgtaccagcccctttcccgaaagtttactgagtggtgttacggtcctgtgcgggtatcgctgtacgacctgtcctctgtggacagctgggaaaagaactcggtgctggagatcatcgcttttcattgcaagagcccgaaccggcaccgcatggtggttttagaaccactgaacaagcttctgcaggagaaatgggatcggctcgtctcaagattcttcttcaacttcgcctgctacttggtctacatgttcatcttcaccgtcgttgcctaccaccagccttccctggatcagccagccatcccctcatcaaaagcgacttttggggaatccatgctgctgctgggccacattctgatcctgcttgggggtatttacctcttactgggccagctgtggtacttttggcggcggcgcctgttcatctggatctcattcatggacagctactttgaaatcctctttctccttcaggctctgctcacagtgctgtcccaggtgctgcgcttcatggagactgaatggtacctacccctgctagtgttatccctagtgctgggctggctgaacctgctttactacacacggggctttcagcacacaggcatctacagtgtcatgatccagaaggtcatccttcgagacctgctccgtttcctgctggtctacctggtcttccttttcggctttgctgtagccctagtaagcttgagcagagaggcccgaagtcccaaagcccctgaagataacaactccacagtgacggaacagcccacggtgggccaggaggaggagccagctccatatcggagcattctggatgcctccctagagctgttcaagttcaccattggtatgggggagctggctttccaggaacagctgcgttttcgtggggtggtcctgctgttgctgttggcctacgtccttctcacctacgtcctgctgctcaacatgctcattgctctcatgagcgaaactgtcaaccacgttgctgacaacagctggagcatctggaagttgcagaaagccatctctgtcttggagatggagaatggttactggtggtgccggaggaagaaacatcgtgaagggaggctgctgaaagtcggcaccaggggggatggtacccctgatgagcgctggtgcttcagggtggaggaagtaaattgggttgcttgggagaagactcttcccaccttatctgaggatccatcagggccaggcatcactggtaataaaaagaacccaacctctaaaccggggaagaacagtgcctcagaggaagaccatctgccccttcaggtcctccagtccccctgatggcccagatgcagcagcaggctggcaggatggagtagggaatcttcccagccacaccagaggctactgagttttggtggaaatataaatatttttttgcataaccaaaaaaaaaaaaaaaaaaaaaaaaaaaaaaaagg

SEQ ID NO: 3 - DNA, 2440 bp, Mus musculus
gatctcaaggcaaggactgccaccaccatctggaacctgccagcatatgccttaggctccagcaatgaatgcccactccaaggagatggtgcccctcatgggcaaaagaaccacggcacctggcgggaaccctgttgtactgacagagaagaggccagcagatctcacccccaccaagaagagtgcacacttcttcctggagatagaaggatttgagcccaaccccacggtcaccaagacctctccacccatcttctccaagccgatggactccaacatccggcagtgcctctctggcaactgtgatgacatggactctccccagtctcctcaggatgatgtgacagagaccccatccaatcccaacagtccgagcgcaaacctggccaaggaagaacagaggcagaagaagaagcgactgaagaagcgcatcttcgcggctgtgtccgagggctgcgtggaggagctgcgggaactcctacaggatctgcaggacctctgcaggaggcgccgcggcctggatgtgcctgacttcctcatgcacaagctgacagcctcagacaccgggaagacctgcctgatgaaggctttgctcaacatcaatcccaacaccaaagagatcgtgcggattctgcttgccttcgctgaggagaacgacatcctggacaggttcatcaacgctgagtacacggaagaggcctatgaagggcagacagcgctgaacatcgccatcgagcggcgccagggagacatcacagcagtgcttatagcagcgggtgctgacgtcaatgctcacgccaagggggtcttcttcaaccccaaataccagcatgaaggcttctattttggcgagacacccctggctttggcagcgtgtactaaccagcctgagattgtgcagctgctgatggagaatgagcagacagacatcacttcccaggattcccggggaaacaacatcctgcacgcgctggtgacagtggctgaggacttcaagactcagaatgacttcgttaagcgcatgtatgacatgatcctgctgaggagtggcaactgggagctggagaccatgcgcaacaacgatgggctcacaccactgcagctggctgccaagatgggcaaggctgagatcctgaagtacatcctcagccgcgagatcaaggagaagcctctccggagcttgtccaggaagttcacggactgggcgtatgggcctgtgtcatcctcactctatgacctcaccaatgtagacacaacgacggataactctgtgctggaaatcatcgtctacaacaccaacattgataaccgacatgagatgctgaccctggagcctctgcatacgctgctacacacgaaatggaagaaatttgccaagtacatgttcttcttgtccttctgcttctatttcttctacaacatcaccctgacccttgtctcttactaccgtcctcgggaagatgaggatctcccacaccccttggccctgacacacaaaatgagttggcttcagctcctagggaggatgtttgtcctcatctgggccacatgcatctctgtgaaagaaggcattgccattttcctgctgagaccctccgatcttcagtccatcctgtcagatgcctggtttcactttgtcttttttgtccaagctgtacttgtgatactgtctgtattcttgtacttgtttgcctacaaagaatacctcgcctgcctcgtgctggccatggccctgggctgggcgaacatgctctactacacgagaggcttccagtctatgggcatgtacagcgtcatgatccagaaggtcattttgcatgatgtcctcaagttcttgtttgtttacatcctgttcttacttggatttggagtagcgctggcctcactgattgagaagtgctccaaggacaaaaaggactgcagttcctatggcagcttcagcgacgcggtgctggagctcttcaagctcaccataggcctgggcgacctgaacatccagcagaactccacctaccccatcctctttctcttcctactcatcacctatgtcatcctcaccttcgtcctcctcctcaacatgctcatcgccctgatgggggagacggtggagaacgtctccaaagaaagtgagcggatctggcgcttgcagagagccaggaccatcttggagtttgagaaaatgttaccagaatggctgagaagcagattccgcatgggcgagctgtgcaaagtagcagatgaggacttccggctgtgtctgcggatcaacgaggtgaagtggacggaatggaaaacacacgtgtccttccttaatgaagacccgggacccataagacggacagcagatttaaacaagattcaagattcttccaggagcaatagcaaaaccaccctctatgcgtttgatgaattagatgaattcccagaaacgtcggtgtag

SEQ ID NO: 4 - DNA, 3211 bp, Rattus norvegicus
gggaggaggacgcggcgggatcaggaagcggctgcgctgcgcccgcgtcccaagcaggccgagaagtccaaacagatctgctcagggtccagtatggcagatcctggtgatggcccccgtgcagcgcctggggatgtggctgagccccctggagacgagagtggcacttctggtggggaggccttccccctctcttccctggccaacctgtttgagggagaggaaggctcctcttctctttcaccagtggatgctagccgccctgctggccccggggatggacgtccaaacctgcgtatgaagttccagggcgctttccgcaagggggttcccaaccccattgacctgctggagtccaccctgtatgagtcctcagtagtgcctgggcccaagaaagcgcccatggattcgttgttcgactatggcacttaccggcaccaccccagtgacaacaagagatggaggaggaaggtcgtagagaagcagccacagagccccaaagctcccgccccccagccaccccccatcctcaaagtcttcaaccggcccatcctctttgacatcgtgtcccggggctccactgccgacctggacggactgctctcctacttgctgacccacaagaagcgcctgactgatgaggagttccgggaaccatccacagggaagacctgcctgcccaaggcacttctgaacttaagcaatggccgaaacgacaccatcccagtgttgctggacattgcggaacgcacgggcaacatgcgggagttcatcaactcgcccttcagagacatctactaccgagggcagacggcactgcacatcgccattgaacggcgctgcaagcattacgtggagctcctggtggcccagggagccgatgtgcacgcgcaggcccgagggcggttcttccagcccaaggatgagggtggctacttctactttggggagctgcccttgtccttggcagcctgcaccaaccagccgcacatcgtcaactacctgacagagaaccctcacaagaaagccgatatgaggcgacaggactccagaggcaacacggtgctccacgcgctggtggccatcgctgacaacacccgagagaacaccaagtttgtcaccaagatgtatgacctgttgcttctcaagtgctcccgcctcttcccagacagcaacctggagactgtgcttaacaatgacggtctttcgcccctcatgatggctgccaagactggcaagatcggggtctttcagcacatcatccgacgggaggtgacagatgaggacacacggcacctgtctcgcaagttcaaggactgggcctacgggcctgtgtattcttctctctacgacctctcctccctggatacgtgcggggaggaagtgtccgtgctggagatcctggtttacaacagcaagatcgagaaccgccatgagatgctggctgtggagcccattaacgaactgctgagggacaagtggcgtaagttcggggccgtgtccttctacatcaacgttgtctcctatctgtgtgccatggtcatcttcaccctcacagcctactatcagccactggagggcacgccaccctacccttaccgtaccacggtggactacctgaggctggctggtgaggtcatcacgctcctcacaggagtcctgttcttctttaccagtatcaaagacttgttcatgaagaaatgccctggagtgaattctctcttcgtcgatggctccttccagttgctctacttcatctactcagtgctggtggttgtgtctgcggcgctctacctggcagggatcgaggcctatctggctgtgatggtctttgccctggtcctgggctggatgaatgccctttacttcacccgtgggctgaagctgacagggacctacagcatcatgattcagaagatcctcttcaaagatctcttccgctttctgctggtctacctgctttttatgattggctatgcctcagctctggtcaccctcctgaatccgtgcaccaacatgaaggtctgtaacgaggaccagagcaactgcacggtgccctcataccccgcgtgccgggacagcgagaccttcagcgccttcctactggacctcttcaagctcaccatcggcatgggcgacctggagatgctgagcagcgctaagtaccccgtggtcttcattctcctgctggttacctacatcatcctcaccttcgtgctcctgctgaacatgctcatcgccctcatgggtgagaccgtgggccaggtgtccaaggagagcaagcacatctggaagctgcagtgggccaccaccatcctggacatcgagcgctccttccctgtgttcctgaggaaggccttccgctccggagagatggtgacagtgggcaagagctcggatggcactccagaccgcaggtggtgcttcagggtggacgaggtgaactggtctcactggaaccagaacctgggcatcattaacgaggaccccggcaagagcgagatctaccagtactatggcttctcccataccatggggcgcctccgcagggatcgctggtcctcagtggtgccccgcgtggtggagctgaacaagaactcaggcacagatgaagtggtggtccccctggataacctagggaaccccaactgtgacggccaccagcaaggttatgctcccaagtggagggcggaggacgcaccactgtaggggccatgccagggctggggtcaatggcccaggcttggcccttgctcccacctacatttcagcatctgtcctgtgtcttcccacacccacacgtgacctcggaggtgagggcctctgtggagactctggggaggccccaggaccctctggtccccacaaagacttttgctcttatttctactcctccccacatgggggacggggctcctggccacctgtctcactcccatggagtcacctaagccagctcagggcccctccactcacagggctcaggcccctgtccctcttgtgcactatttattgctctcctcaggaaaatgacatcacaggagtctacctgcagctggaacctggccagggctgaggctcatgcagggacactgcagccctgacccgctgcagatctgacctgctgcagcccgggctagggtgggtcttctgtactttgtagagatcggggctgttggtgctcaataaatgtttgtttattctcggtgga

SEQ ID NO: 5 - DNA, 3869 bp, Mus musculus
tcctccctcctccagtgagctaagagacaagcaggctctttgaggagagagaagctcttggctgattgagcagctccacgtcctggctgtcccggagcttgatacatagaaaagactgacctcagatacacagagatccttctgcttctgtctcccaagtgctgggatcacaggcaagatgtccttcgagggagccaggctcagcatgaggagccgcagaaatggtactatgggcagcacccggaccctgtactccagtgtatctcggagcacagacgtgtcctacagtgacagtgatttggtgaattttattcaggcaaattttaaaaaacgagaatgtgtcttctttaccagagactccaaggccatggagaacatatgcaagtgtggttatgcccagagccagcacatcgaaggcacccagatcaaccaaaatgagaagtggaactacaaaaaacataccaaggagtttccaacagacgccttcggggacattcagtttgagactctggggaagaaaggcaagtacttacgcttgtcctgtgacaccgactctgaaactctctacgaactgctgacccagcactggcacctcaaaacacccaacctggtcatttcagtgacgggtggagccaaaaactttgctttgaagccacgcatgcgcaagatcttcagcaggctgatttacatcgcacagtctaaaggtgcgtggattctcactggaggcactcactacggcctgatgaagtacataggcgaggtggtgagagacaacaccatcagcaggaactcagaagagaacatcgtggccattggcatcgcagcatggggcatggtctccaacagggacaccctcatcaggagctgtgatgatgagggacatttttcagctcaatacatcatggatgactttaccagagaccctctatacatcctggacaacaaccatacccacctgctgcttgtggacaacggttgtcatggacaccccacagtggaagccaagctccggaatcagctggaaaagtacatctctgagcgcaccagtcaagattccaactatggtggtaagatccccatcgtgtgttttgcccaaggaggtggaagagagactctaaaagccatcaacacctctgtcaaaagcaagatcccttgtgtggtggtggaaggctcggggcagattgctgatgtgatcgccagcctggtggaggtggaggatgttttaacctcttccatggtcaaagagaagctggtacgctttttaccacgcactgtgtcccggctgcctgaagaggaaattgagagctggatcaaatggctcaaagaaattcttgagagttctcacctactcacagtaattaagatggaagaggctggagatgagattgtgagcaacgccatttcctatgcgctgtacaaagccttcagcactaatgagcaagacaaggacaactggaatggacagctgaagcttctgctggagtggaaccagttggaccttgccagtgatgagatcttcaccaatgatcgccgctgggagtctgccgaccttcaggaggtcatgttcacggctctcataaaggacagacccaagtttgtccgcctctttctggagaatggcctgaatctgcagaagtttctcaccaatgaagtcctcacagagctcttctccacccacttcagcaccctagtgtaccggaatctgcagatcgccaagaactcctacaatgacgcactcctcacctttgtctggaagttggtggcaaacttccgtcgaagcttctggaaagaggacagaagcagcagggaggacttggatgtggaactccatgatgcatctctcaccacccggcacccgctgcaagctctcttcatctgggccattcttcagaacaagaaggaactctccaaggtcatttgggagcagaccaaaggctgtactctggcagccttgggggccagcaagcttctgaagaccctggccaaagttaagaatgatatcaacgctgctggggaatcggaggaactggccaatgaatatgagacccgagcagtggagttgttcaccgagtgttacagcaatgatgaagacttggcagaacagctactggtctactcctgcgaagcctggggtgggagcaactgtctggagctggcagtggaggctacagatcagcatttcatcgctcagcctggggtccagaatttcctttctaagcaatggtatggagagatttcccgagacacgaagaactggaagattatcctgtgtctattcatcatccccttagtgggctgtggcctcgtatcatttaggaagaaacccattgacaagcacaagaagctgctgtggtactatgtggccttcttcacgtcgcccttcgtggtcttctcctggaacgtggtcttctacatcgccttcctcctgctgtttgcctatgtgctgctcatggacttccactcagtgccacacacccccgagctgatcctctacgccctggtcttcgtcctcttctgtgatgaagtgaggcagtggtacatgaacggagtgaattatttcaccgacctatggaacgttatggacaccctgggactcttctacttcatagcgggtattgtattccggctccactcttctaataaaagctcgttgtactctgggcgcgtcattttctgtctggattacattatattcacgctaaggctcatccacattttcaccgtcagcaggaacttgggacccaagattataatgctgcagcggatgctgatcgacgttttcttcttcctgttcctctttgctgtgtggatggtggcctttggcgtggccagacaggggatcctaaggcaaaatgaacagcgctggagatggatcttccgctctgtcatctatgagccctacctggccatgtttggccaggttcccagtgacgtggatagtaccacatatgacttctcccactgtaccttctcgggaaatgagtccaagccactgtgtgtggagctggatgagcacaacctgccccgcttccctgagtggatcaccattccgctggtgtgcatctacatgctctccaccaatatccttctggtcaacctcctggtcgccatgtttggctacacggtaggcattgtacaggagaacaacgaccaggtctggaaattccagcggtacttcctggtgcaggagtactgcaaccgcctaaacatccccttccccttcgttgtcttcgcttatttctacatggtggtgaagaagtgtttcaaatgctgctgtaaagagaagaatatggagtctaatgcctgctgtttcagaaatgaggacaatgagactttggcgtgggagggtgtcatgaaggagaattaccttgtcaagatcaacacgaaagccaacgacaactcagaggagatgaggcatcggtttagacaactggactcaaagcttaacgacctcaaaagtcttctgaaagagattgctaataacatcaagtaaggctggcgatgcttgtggggagaaaccaaatcacaatgaggtcacagcaaccacctggatgtggaggctcatgggacactgatggacagtactgctaatgacttctaaaggagacattttcaggtccctgagcacagggtggatgactcttagtcaccctcaagggcataggtcagggagcaaagtgtacagaggactttacacctgaagaggggtgcaaaggaccatgttcttctgtgaaggtgcctgtgttttctgcatctcagagccttgtcctgatgctgagggattaagtgttgacactcctttcccacgactgtgactctggccctgattttatacttatactgcaaaaaaaaaaaaaaaaaaaaaaaaa

SEQ ID NO: 6 - DNA, 4263 bp, Mus musculus
gcgccagccggcgtccaggtggagtcaatgaagcgcggcttgaggaggattctgctcccggaggaaaggaaggaggtccagggcgttgtctatcgcggcgtcggggaagacatggactgctccaaggaatcctttaaggtggacattgaaggagatatgtgtagattagaagacttcatcaagaaccgaagaaaactaagcaaatatgaggatgaaaatctctgtcctctgcatcacgcagcagcagaaggtcaagttgaactgatggaactgatcatcaatggttcttcgtgtgaagtgctgaatataatggatggttatggaaataccccactgcattgtgctgcagaaaaaaatcaagttgaaagtgtaaagtttcttctcagccaaggagcaaatccaaacctccgaaatagaaacatgatgtcaccccttcacatagctgtgcatggcatgtacaacgaagtgatcaaggtgttgactgagcacaaggccactaacatcaatttagaaggagagaatgggaacacggctttgatgtccacgtgtgccaaagacaacagtgaagctttgcaaattttgttagaaaaaggagctaagctgtgtaaatcaaataagtggggagactaccctgtgcaccaggcagcattttcaggtgccaaaaaatgcatggaattaatcttagcatatggtgaaaagaacggctacagcagggagactcacattaattttgtgaatcacaagaaagccagccctctccacctagcagttcaaagcggagacttggacatgattaagatgtgcctggacaacggtgcacacatcgacatgatggagaatgccaaatgcatggccctccattttgctgcaacccagggagccactgacatcgttaagctcatgatctcatcctataccggaagtagtgatattgtgaatgcagttgatggcaatcaggagaccctgcttcacagagcctcgttatttgatcaccatgacctggcagaatacctaatatcagtgggagcagacatcaacagcactgattctgaaggacgctctccacttattttagcaacagcttctgcatcctggaacattgtgaatttgctcctctgtaaaggtgccaaagtagacataaaagatcatcttggacgtaactttttgcatttgactgtgcagcagccttatggactaagaaatttgcggcctgagtttatgcagatgcaacacatcaaagagctggtgatggatgaagacaatgacggatgcacacctctccattatgcctgtaggcagggggttcctgtctctgtaaataacctccttggcttcaatgtgtccattcatagcaaaagtaaagataagaagtcgcccctgcattttgcagccagttatgggcgcatcaatacatgtcagagacttctgcaagacataagtgatacgaggcttttgaatgaaggggatctccatgggatgacccctctccacctggcagcaaaaaatgggcatgataaagtcgttcaactccttctgaagaaaggggccttatttctcagtgaccacaatggctggactgctttgcatcacgcctccatgggtgggtacactcagaccatgaaggtcattcttgatactaacttgaaatgcacagaccgactagatgaagaagggaacacagcactccactttgcagcacgggaaggccatgccaaggctgttgcaatgcttttgagctacaatgctgacatcctcctgaacaagaagcaagcttcctttctgcatattgccctgcacaataagcgcaaggaagtggttctcacaaccatcagaaataaaagatgggatgagtgtcttcaagttttcactcataattctccaagcaatcgatgtccaatcatggagatggtagaatacctccccgagtgcatgaaagttcttttagatttctgcatgataccttccacagaagacaagtcctgtcaagactaccatattgagtataatttcaagtatctccaatgcccattatccatgaccaaaaaagtagcacctacccaggatgtggtatatgagcctcttacaatcctcaatgtcatggtccaacataaccgcatagaactcctcaaccaccctgtgtgtagggagtacttactcatgaaatggtgtgcctatggattcagagcccatatgatgaacctaggatcttattgtcttggtctcatacccatgacccttcttgttgtcaaaatacagcctggaatggccttcaattctactggaataatcaatggaactagtagtactcatgaggaaagaatagacactctgaattcatttccaataaaaatatgtatgattctagtttttttatcaagtatatttggatattgcaaagaagtgatccaaattttccaacagaaaaggaattacttcctggattacaacaatgctctggaatgggttatctatacaactagtatcatcttcgtgttgcccttgttcctcaacatcccagcgtatatgcagtggcaatgtggagcaatagcgatattcttctactggatgaacttcctactgtatcttcaaaggtttgagaactgtggaattttcattgttatgttggaggtgatttttaaaacattgctgagatcgaccggagtgtttatcttcctcctactggcttttggcctcagcttttatgttctcctgaatttccaagatgccttcagcaccccattgctttccttaatccagacattcagtatgatgctaggagacatcaattatcgagatgccttcctagaaccattgtttagaaatgagttggcatacccagtcctgacctttgggcagcttattgccttcacaatgtttgtcccaattgttctcatgaacttactgattggcttggcggttggggacattgctgaggtccagaagcatgcgtcattgaagaggattgctatgcaggtggaacttcataccaacttagaaaaaaagctgccactctggtacttacgcaaagtggatcagaggtccaccatcgtgtatccaaatagacccaggcacggcaggatgctacggttttttcattactttcttaatatgcaagaaacacgacaagaagtaccaaacattgacacatgcttggaaatggaaatattgaaacagaaatatcggctgaaggacctcacttccctcttggaaaagcagcatgagctcatcaaactcatcatccagaagatggagatcatctcagagacagaagatgaagataaccattgctctttccaagacaggttcaagaaggagaggctggaacagatgcacagcaagtggaattttgtcttaaacgcagttaagactaaaacacattgttctattagccacccggacttttagttctgtgtcttatgggagtgggagactgctttacatacttatttcagtgaatttcagtttggaaaagagcaaagaaacagaaagttgactaacattgctgcatggagatcctagttcctgcaacctcacccatacatatgctcatatttcctgtcaattactatgtattgagaagatcctttctgacatgttcaatttgaacatgaaggatagtctctttcgagtgaataaaaaccagggttgttggaatgcatattatggaggataagaattaatgtaactattaaggcagaacacaactacataatacaagatgcatataattccaagtattatatttaatctcctaccatgttaaaccttcctgtgttataacctgtctgggacactataatctctgttcctactatgattagatcatagtctcaccctcctcgtcccatcacacatgacatcattttgagccacatgacagaagtcctagttagtagactgtgataagtatgaatgttacaatagaaatgtgttcccttagtgttcatcagttgtgatggtttaaatgagaaacgttgcccacagactcatacatttaaacccttagtcccagttgttgctgctgcttaggggggccacacagccttgcttgctctctcctttctgagtgtggagagaaatgtgatcagtaagactcctgctcctgctgccatgctctttattccattatggacttcttctgaaactgcaagcagaaattcactgttccttcctcaaatttcttttggtcatggtattatatcatagcaacagaaactaacttatgtaccaatggtcttaataaagaataaagcctgtacagtc

(19) United States
(12) Patent Application Publication
(10) Pub. No.: US 2010/0137260 A1
(43) Pub. Date: Jun. 3, 2010

(54) COMPOUND FOR INHIBITING TRPV3 FUNCTION AND USE THEREOF

(75) Inventors: Sun Wook HWANG, Seoul (KR); Sang-Soo Bang, Gyeonggi-do (KR)

Correspondence Address: GREENLEE WINNER AND SULLIVAN P C, 4875 PEARL EAST CIRCLE, SUITE 200, BOULDER, CO, 80301, US

(73) Assignee: Korea University Industry and Academic Collaboration Foundation, Seoul (KR)

(21) Appl. No.: 12/388,061

(22) Filed: Feb. 18, 2009

(30) Foreign Application Priority Data
Dec. 2, 2008 (KR) 10-2008-0121094

(51) Int. Cl. A61K 31/66; A61P 17/06

(52) U.S. Cl. 514/143

(57) ABSTRACT

The present invention relates to a TRPV3 (transient receptor potential vanilloid 3) activity inhibitor, more precisely to a method for inhibiting TRPV3 activity including the step of treating isopentenyl pyrophosphate and a method for treating skin disease containing the step of administering isopentenyl pyrophosphate to a subject with skin disease or applying the same on the skin of the subject. Isopentenyl pyrophosphate of the present invention controls increase of sensory cell reactivity to current or migration and proliferation of skin cells induced by TRPV3, so that it can be effectively used for the development of a pain reliever or a therapeutic agent for skin disease.

### CROSS-REFERENCE TO RELATED APPLICATIONS

This application claims priority under 35 USC 119 (a)-(d) to Korea Application No. 10-2008-0121094 filed on Dec. 2, 2008, the contents of which are hereby incorporated by reference in their entirety.

### BACKGROUND OF THE INVENTION

The present invention relates to a TRPV3 activity inhibitor, more precisely isopentenyl pyrophosphate, a compound for suppressing TRPV3 mediated biological phenomena such as pain and skin growth by inhibiting TRPV3 activity and a novel use of the same.

TRPV3 (transient receptor potential vanilloid 3), a high temperature receptor in human, was first found in 2003 owing to the studies in the fields of human physiology and pharmacology. TRPV3 was presumed to play an essential role in maintaining survival system in various tissues. In particular, TRPV3 is expressed in skin cells and peripheral sensory nerve cells which recognize foreign stimuli. TRPV3 belongs to thermoTRP family (temperature-sensitive transient receptor potential ion channels) that is the pain receptor family recognizing temperature and painful stimuli. In 2005, pain mediation mechanism of TRPV3 was scientifically disclosed by the behavior research with TRPV3 knock-out animals. Many researchers expect that human pain mechanism will be disclosed by understanding the functions of TRPV3, the pain receptor, and finally the goal of relieving pain will be achieved by the development of a TRPV3 regulator. To examine TRPV3 functions and develop a TRPV3 regulator, a TRPV3 specific activator that only works for TRPV3 without affecting any other TRP receptors is required.

To understand basic techniques used for the development of a TRPV3 specific inhibitor, it is important to understand the characteristics of TRPV3. TRPV3 is an ion channel and its activation makes cations migrate into sensory neurons or skin cells, stimulating intracellular signal transduction system. For skin, this calcium signal transduction system regulates cell growth and differentiation and at last determines skin cell destiny. One of the techniques to measure TRPV3 activation is patch-clamp electrophysiological technique measuring the changes of membrane currents after amplifying thereof. And another technique to measure TRPV3 activation is to measure intracellular calcium level based on the fact that TRPV3 is involved in the migration of cations such as calcium ions. The first technique is superior in sensitivity to the second one, but the second technique is superior in high speed to the first one, so that they are complementary to each other. Such techniques to measure TRPV3 activation can be executed by the support of animal neuron culture technique, cell line culture technique, TRPV3 DNA control and transfection techniques. Various TRPV3 specific inhibitor candidates are administered to TRPV3 over-expressing cells and then inhibiting effect of TRPV3 activation therein is measured to select a proper TRPV2 inhibitor and determine its capacity.

A TRPV3 specific inhibitor is an essential element to measure TRPV3 activation for further development of a TRPV3 regulator. However, no reports have been made so far in relation to a TRPV3 specific inhibitor. The known TRPV3 inhibitor so far is ruthenium red, but this is the material that inhibits all the calcium channels and does not have specificity to TRPV3.

Wound is healed by the increase of skin cell migration and growth. So, when skin cell migration and growth is inhibited, skin disease caused by over-growth of cells such as psoriasis, lichen planus, keratosis, basal cell carcinoma, hypersensitive dermatitis, atopic dermatitis, seborrheic dermatitis, and keloid can be treated [Pani B & Singh B B, Cell Mol Life Sci. 65(2):205-211, 2008 (keloid, hypersensitive dermatitis, hereditary dermatitis, etc); Hanifin J M, J Invest Dermatol. 2008 (atopy, seborrheic dermatitis); Zhao Y et al., J Invest Dermatol. 128(9):2190-2197, 2008 (atopy, psoriasis); Bovenschen H J et al., Br J Dermatol. 153(1):72-78, 2005 (atopy, lichen planus); Brennan D et al., J Cell Sci. 120(Pt 5):758-771, 2007 (basal cell carcinoma, keratosis); Bhoumik A et al., Proc Natl Acad Sci USA. 105(5):1674-1679, 2008 (basal cell carcinoma); Teh M T et al., J Cell Sci. 120(Pt 2):330-339, 2007 (basal cell carcinoma); Birnbaum R Y et al., Nat Genet. 38(7):749-751, 2006 (keratosis, lichen planus); Lim C P et al., Oncogene. 25(39):5416-5425, 2006 (keloid); Lim C P et al., J Invest Dermatol. 2008(keloid); Korean Patent No 10-0771523 (psoriasis, hypersensitive dermatitis, lichen planus, basal cell carcinoma)]. For example, calcipotriol (product name: DAIVONEX) inhibits proliferation of keratinocytes, the myoblasts of HaCat skin cells, so that it is believed to have treatment effect on the propagative skin disease such as psoriasis. In fact, it has been sold as a drug for psoriasis treatment.

The present inventors constructed a cell line expressing TRPV3 and treated the cell line with isopentenyl pyrophosphate and camphor known as a TRPV3 inhibitor. Then, responses therein were compared. At last the present inventors completed this invention by confirming that isopentenyl pyrophosphate inhibited TRPV3 activity and thus it could be effectively used as an inhibitor of TRPV3 mediated biological phenomena such as pain and skin growth.

### SUMMARY OF THE INVENTION

It is an object of the present invention to provide a method for inhibiting TRPV3 activity using isopentenyl pyrophosphate.

It is another object of the present invention to provide a method for screening a TRPV3 activity inhibitor using isopentenyl pyrophosphate.

It is also an object of the present invention to provide a method for inhibiting pain containing the step of administering a pharmaceutically effective dose of isopentenyl pyrophosphate to a subject.

It is further an object of the present invention to provide a method for relieving pain containing the step of providing functional food containing isopentenyl pyrophosphate as an active ingredient to a subject.

It is also an object of the present invention to provide a method for treating skin disease containing the step of administering a pharmaceutically effective dose of isopentenyl pyrophosphate to a subject or applying the same on the skin of the subject.

To achieve the above objects, the present invention provides a method for inhibiting TRPV3 activity containing the step of treating isopentenyl pyrophosphate to isolated sensory neurons or skin cells expressing TRPV3.

The present invention also provides a method for screening a TRPV3 activity inhibitor comprising the following steps:

1) constructing a transformant by transfecting a host cell with a plasmid harboring the polynucleotide encoding TRPV3;

2) treating the transformant with TRPV3 specific activator and TRPV3 activity inhibitor candidates as the experimental group, and treating the transformant with TRPV3 specific activator and isopentenyl pyrophosphate as the control;

3) measuring TRPV3 ion channel activities in the experimental group and in the control group of step 2); and

4) comparing the results of step 3) and selecting TRPV3 activity inhibitor candidates from the experimental group that demonstrated lower or similar TRPV3 ion channel activity, compared with the control.

The present invention further provides a method for inhibiting pain containing the step of administering a pharmaceutically effective dose of isopentenyl pyrophosphate to a subject.

The present invention also provides a method for treating skin disease containing the step of administering a pharmaceutically effective dose of isopentenyl pyrophosphate to a subject or applying the same on the skin of the subject.

Isopentenyl pyrophosphate of the present invention controls increase of sensory cell reactivity to current or migration and growth of skin cells induced by TRPV3, so that it facilitates the development of an effective pain inhibitor or a treatment agent for skin disease.

### BRIEF DESCRIPTION OF THE DRAWINGS

The application of the preferred embodiments of the present invention is best understood with reference to the accompanying drawings, wherein:

FIG. 1 is a diagram illustrating that TRPV3 specific activity induced by camphor in TRPV3 cell line was inhibited by isopentenyl pyrophosphate:

a: Fluo-3 calcium imaging; and,

b: whole cell voltage clamp technique

FIG. 2 is a diagram illustrating that TRPV3 activity was specifically inhibited by isopentenyl pyrophosphate in TRPV3 cell line.

FIG. 3 is a diagram illustrating that cell migration and proliferation were inhibited by isopentenyl pyrophosphate.

### DETAILED DESCRIPTION OF THE INVENTION

Hereinafter, the present invention is described in detail.

The present invention provides a method for inhibiting TRPV3 activity containing the step of treating isopentenyl pyrophosphate to isolated sensory neurons or skin cells expressing TRPV3.

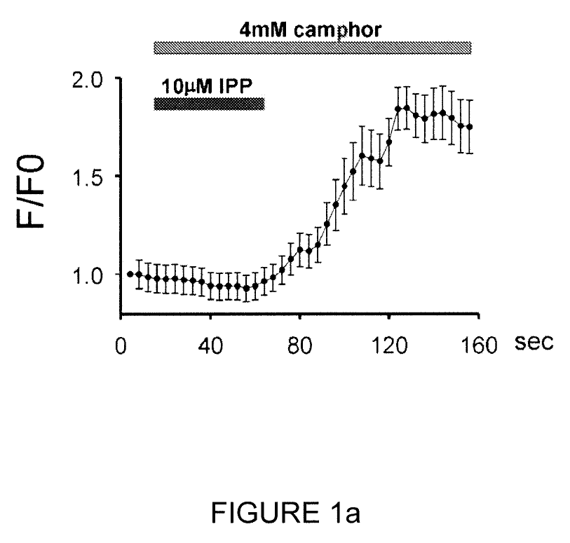

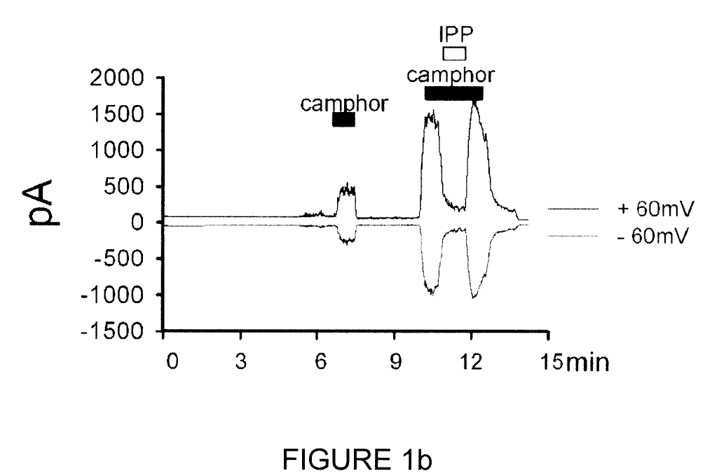

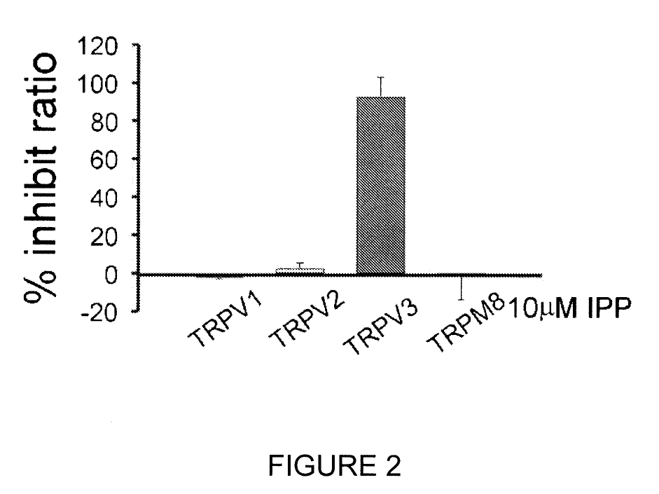

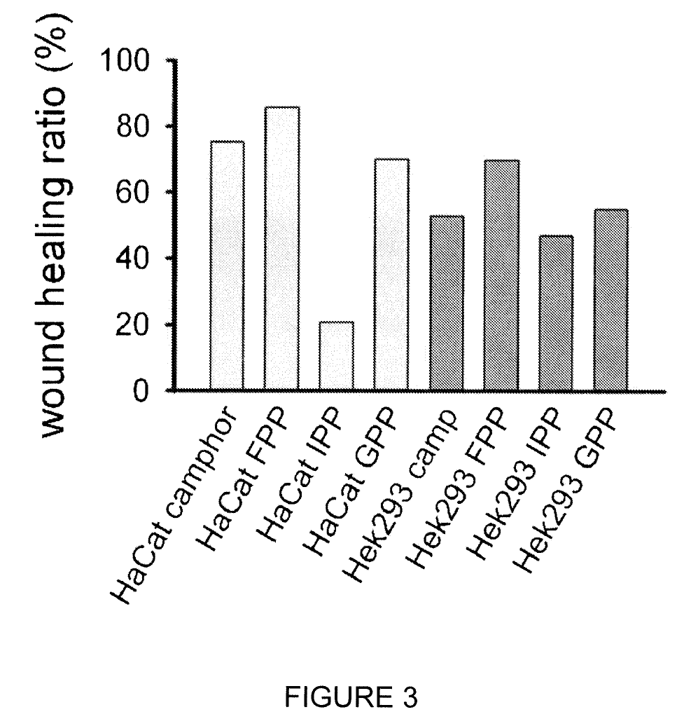

In a preferred embodiment of the present invention, it was confirmed that TRPV3 activity induced by camphor was inhibited by isopentenyl pyrophosphate by whole cell voltage clamp technique, a kind of patch clamp techniques, and calcium imaging, a technique to detect intracellular calcium level changes (see FIG. 1a and FIG. 1b). Inhibiting effect of isopentenyl pyrophosphate was observed only in the transformed cell line expressing TRPV3, among the transformed cell lines expressing TRPV1, TRPV2, TRPV3 and TRPM8 (transient receptor potential cation channel, subfamily M, member 8) (see FIG. 2). In addition, in a preferred embodiment of the present invention, isopentenyl pyrophosphate was confirmed to inhibit HaCat skin cell migration and proliferation (see FIG. 3). Therefore, the said isopentenyl pyrophosphate can be effectively used for inhibiting TRPV3 activity.

Isopentenyl pyrophosphate of the present invention can be formulated for oral administration, for example powders, granules, tablets, capsules, suspensions, emulsions, syrups and aerosols, and for parenteral administration, for example external use, suppositories and sterile injections, etc.

Solid formulations for oral administration are powders, granules, tablets, capsules, soft capsules and pills. Liquid formulations for oral administration are suspensions, solutions, emulsions and syrups, and the above-mentioned formulations can contain various excipients such as wetting agents, sweeteners, aromatics and preservatives in addition to generally used simple diluents such as water and liquid paraffin. For formulations for parenteral administration, powders, granules, tablets, capsules, sterilized suspensions, liquids, water-insoluble excipients, suspensions, emulsions, syrups, suppositories, external use such as aerosols and sterilized injections can be prepared by the conventional method, and preferably skin external pharmaceutical compositions such as creams, gels, patches, sprays, ointments, plasters, lotions, liniments, pastes or cataplasms can be prepared, but not always limited thereto. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

The present invention also provides a method for screening a TRPV3 activity inhibitor comprising the following steps:

1) constructing a transformant by transfecting a host cell with a plasmid harboring the polynucleotide encoding TRPV3;

2) treating the transformant with TRPV3 specific activator and TRPV3 activity inhibitor candidates as the experimental group, and treating the transformant with TRPV3 specific activator and isopentenyl pyrophosphate as the control group;

3) measuring TRPV3 ion channel activities in the experimental group and in the control group of step 2); and

4) comparing the results of step 3) and selecting TRPV3 activity inhibitor candidates from the experimental group that demonstrated lower or similar TRPV3 ion channel activity, compared with the control.

In a preferred embodiment of the present invention, it was confirmed that TRPV3 activity induced by camphor known as a TRPV3 activator was inhibited specifically by isopentenyl pyrophosphate (see FIG. 1 and FIG. 2). It was also confirmed that HaCat skin cell migration and proliferation were also inhibited by isopentenyl pyrophosphate (see FIG. 3). So, the said isopentenyl pyrophosphate can be effectively used for the screening of a TRPV3 activity inhibitor.

The host cell herein is preferably any cell line that can be used for the study of calcium channel activity and high throughput screening, for example HEK, CHO, HeLa, RBL-2H3, and HaCat, but not always limited thereto.

The TRPV3 specific activator of step 2) is camphor.

The measuring of ion channel activity of step 3) can be performed by whole cell voltage clamp technique or calcium imaging.

The preferable concentration of isopentenyl pyrophosphate is 0.1-100 μM.

The present invention also provides a method for inhibiting pain containing the step of administering a pharmaceutically effective dose of isopentenyl pyrophosphate to a subject.

In a preferred embodiment of the present invention, it was confirmed that TRPV3 activity induced by camphor known as a TRPV3 activator was inhibited specifically by isopentenyl pyrophosphate (see FIG. 1 and FIG. 2). So, the said isopentenyl pyrophosphate can be effectively used for inhibiting pain.

The pain herein is mediated by TRPV3 activity.

The subject herein is one of vertebrates and preferably mammals and more preferably selected from such test animals as rats, rabbits, guinea pigs, hamsters, dogs and cats, and most preferably apes such as chimpanzees and gorillas.

Isopentenyl pyrophosphate of the present invention can be provided as a pharmaceutical composition. The composition can include, in addition to isopentenyl pyrophosphate, one or more effective ingredients having the same or similar function to isopentenyl pyrophosphate. The composition of the present invention preferably includes isopentenyl pyrophosphate by 0.0001-10 weight % and more preferably 0.001-1 weight % for the total weight of the composition.

The composition of the present invention can additionally include generally used carriers, excipients, disintegrating agents, sweetening agents, lubricants, flavors and diluents. The carriers, excipients and diluents are exemplified by lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil. The disintegrating agent is exemplified by sodium carboxy methyl starch, crospovidone, croscarmellose sodium, alginic acid, calcium carboxymethyl cellulose, sodium carboxymethyl cellulose, chitosan, guar gum, low-substituted hydroxypropyl cellulose, magnesium aluminum silicate, polacrilin potassium, etc.

The pharmaceutical composition of the present invention can additionally include a pharmaceutically acceptable additive, which is exemplified by starch, gelatinized starch, microcrystalline cellulose, lactose, povidone, colloidal silicon dioxide, calcium hydrogen phosphate, lactose, mannitol, taffy, Arabia rubber, pregelatinized starch, corn starch, cellulose powder, hydroxypropyl cellulose, Opadry, sodium carboxy methyl starch, carunauba wax, synthetic aluminum silicate, stearic acid, magnesium stearate, aluminum stearate, calcium stearate, white sugar, dextrose, sorbitol, talc, etc. The pharmaceutically acceptable additive herein is preferably added by 0.1-90 weight part to the pharmaceutical composition.

The composition of the present invention can be administered orally or parenterally. For example the possible administration pathway can be oral administration, rectal administration, intravenous injection, intramuscular injection, hypodermic injection, intrauterine injection or intracerebroventricular injection. The composition for inhibiting pain of the present invention can be administered alone or treated together with surgical operation, hormone therapy, chemo-therapy and biological regulators.

The effective dosage of the pharmaceutical composition of the present invention can be determined by those in the art according to condition and weight of a patient, severity of a disease, type of a drug, administration pathway and duration. Preferably, the composition of the present invention can be administered by 0.0001-100 mg/kg per day, and more preferably by 0.001-100 mg/kg per day. The administration frequency is once a day or a few times a day.

Isopentenyl pyrophosphate of the present invention can be used as food additive. In that case, isopentenyl pyrophosphate can be added as it is or as mixed with other food components according to the conventional method. The mixing ratio of active ingredients can be regulated according to the purpose of use (prevention or health enhancement). In general, to produce health food or beverages, isopentenyl pyrophosphate is added preferably by 0.2-20 weight % and more preferably by 0.24-10 weight %. However, if long term administration is required for health and hygiene or regulating health condition, the content can be lower than the above but higher content can be accepted as well since isopentenyl pyrophosphate has been proved to be very safe.

The health food of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and glucose alcohols such as xylitol, sorbitol and erythritol. Besides, natural sweetening agents such as thaumatin and stevia extract, and synthetic sweetening agents such as saccharin and aspartame can be included as a sweetening agent. The content of the natural carbohydrate is preferably 0.01-0.04 weight part and more preferably 0.02-0.03 weight part in 100 weight part of the health food of the present invention.

The food herein is not limited. For example, isopentenyl pyrophosphate of the present invention can be added to meat, sausages, bread, chocolates, candies, snacks, cookies, pizza, ramyuns, flour products, gums, dairy products including ice cream, soups, beverages, tea, drinks, alcohol drinks and vitamin complex, etc, and in wide sense, almost every food applicable in the production of health food can be included.

In addition to the ingredients mentioned above, the health food of the present invention can include in variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The health food of the present invention can also include natural fruit juice, fruit beverages and/or fruit flesh addable to vegetable beverages. All the mentioned ingredients can be added singly or together. The mixing ratio of those ingredients does not matter in fact, but in general, each can be added by 001-0.1 weight part per 100 weight part of the health food of the present invention.

The present invention also provides a method for treating skin disease containing the step of administering a pharmaceutically effective dose of isopentenyl pyrophosphate to a subject or applying the same on the skin of the subject.

In a preferred embodiment of the present invention, it was confirmed that isopentenyl pyrophosphate inhibited HaCat skin cell migration and proliferation (see FIG. 3). Therefore, isopentenyl pyrophosphate can be effectively used for the treatment of skin disease.

The skin disease herein is resulted from wound healing and over-proliferation of cells, which is selected from the group consisting of psoriasis, lichen planus, keratosis, basal cell carcinoma, hypersensitive dermatitis, atopic dermatitis, seborrheic dermatitis, and keloid.

Isopentenyl pyrophosphate of the present invention can be provided as a pharmaceutical composition. The composition can include, in addition to isopentenyl pyrophosphate, one or more effective ingredients having the same or similar function to isopentenyl pyrophosphate. The pharmaceutical composition of the present invention preferably includes isopentenyl pyrophosphate by 0.0001-10 weight % and more preferably 0.001-1 weight % for the total weight of the composition.

The pharmaceutical composition of the present invention can additionally include generally used carriers, excipients, disintegrating agents, sweetening agents, lubricants, flavors and diluents. The carriers, excipients and diluents are exemplified by lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil. The disintegrating agent is exemplified by sodium carboxy methyl starch, crospovidone, croscarmellose sodium, alginic acid, calcium carboxymethyl cellulose, sodium carboxymethyl cellulose, chitosan, guar gum, low-substituted hydroxypropyl cellulose, magnesium aluminum silicate, polacrilin potassium, etc.

The pharmaceutical composition of the present invention can additionally include a pharmaceutically acceptable additive, which is exemplified by starch, gelatinized starch, microcrystalline cellulose, lactose, povidone, colloidal silicon dioxide, calcium hydrogen phosphate, lactose, mannitol, taffy, Arabia rubber, pregelatinized starch, corn starch, cellulose powder, hydroxypropyl cellulose, Opadry, sodium carboxy methyl starch, carunauba wax, synthetic aluminum silicate, stearic acid, magnesium stearate, aluminum stearate, calcium stearate, white sugar, dextrose, sorbitol, talc, etc. The pharmaceutically acceptable additive herein is preferably added by 0.1-90 weight part to the pharmaceutical composition.

The composition of the present invention can be administered orally or parenterally. For example the possible administration pathway can be oral administration, external application, intraperitoneal injection, rectal administration, hypodermic injection, intravenous injection, intramuscular injection, or intrathoracic injection.

Isopentenyl pyrophosphate of the present invention can be provided in the form of a cosmetic composition for the treatment of skin disease. The skin disease herein is resulted from wound healing and over-proliferation of cells, which is selected from the group consisting of psoriasis, lichen planus, keratosis, basal cell carcinoma, hypersensitive dermatitis, atopic dermatitis, seborrheic dermatitis, and keloid.

The cosmetic composition can be formulated as lotion, ointment, gel, cream, patch or spray, but not always limited thereto. The cosmetic composition of the present invention can additionally include a supplement generally used in the field of skin science such as fatty substance, organic solvent, resolvent, concentrate, gelling agent, softener, antioxidant, suspending agent, stabilizer, foaming agent, odorant, surfactant, water, ionic or non-ionic emulsifying agent, filler, sequestering agent, chelating agent, preserving agent, vitamin, blocker, moisturing agent, essential oil, dye, pigment, hydrophilic or hydrophobic activator, lipid vesicle or other components generally used in a preparation for skin external application. The amount of the above supplement can be determined as generally accepted in the field of skin science.

In addition, isopentenyl pyrophosphate of the present invention can be provided in the form of functional food for the improvement of skin disease. The skin disease herein is resulted from wound healing and over-proliferation of cells, which is selected from the group consisting of psoriasis, lichen planus, keratosis, basal cell carcinoma, hypersensitive dermatitis, atopic dermatitis, seborrheic dermatitis, and keloid.

Isopentenyl pyrophosphate of the present invention can be used as food additive. In that case, isopentenyl pyrophosphate can be added as it is or as mixed with other food components according to the conventional method. The mixing ratio of active ingredients can be regulated according to the purpose of use (prevention or health enhancement). In general, to produce health food or beverages, isopentenyl pyrophosphate is added preferably by 0.2-20 weight % and more preferably by 0.24-10 weight %. However, if long term administration is required for health and hygiene or regulating health condition, the content can be lower than the above but higher content can be accepted as well since isopentenyl pyrophosphate has been proved to be very safe.

The health food of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and glucose alcohols such as xilytole, sorbitol and erythritol. Besides, natural sweetening agents such as thaumatin and stevia extract, and synthetic sweetening agents such as saccharin and aspartame can be included as a sweetening agent. The content of the natural carbohydrate is preferably 0.01-0.04 weight part and more preferably 0.02-0.03 weight part in 100 weight part of the health food of the present invention.

The food herein is not limited. For example, isopentenyl pyrophosphate of the present invention can be added to meat, sausages, bread, chocolates, candies, snacks, cookies, pizza, ramyuns, flour products, gums, dairy products including ice cream, soups, beverages, tea, drinks, alcohol drinks and vitamin complex, etc, and in wide sense, almost every food applicable in the production of health food can be included.

In addition to the ingredients mentioned above, the health food of the present invention can include in variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The health food of the present invention can also include natural fruit juice, fruit beverages and/or fruit flesh addable to vegetable beverages. All the mentioned ingredients can be added singly or together. The mixing ratio of those ingredients does not matter in fact, but in general, each can be added by 001-0.1 weight part per 100 weight part of the health food of the present invention.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### EXAMPLE 1

### Construction of Cell Lines Transfected With TRPV

HEK293T cell line (ATCC CRL-11268) was transiently transfected with plasmid DNA containing polynucleotide encoding rTRPA1 (SEQ. ID. NO: 1), rTRPV2 (SEQ. ID. NO: 2), mTRPV3 (SEQ. ID. NO: 3), rTRPV4 (SEQ. ID. NO: 4), mTRPM8 (SEQ. ID. NO: 5) or mTRPA1 (SEQ. ID. NO: 6).

Particularly, the HEK293T cell line was transiently transfected with 3 μg/35 mm dish of pcDNA3.1 vector (containing polynucleotide encoding hTRPV3, rTRPV2, rTRPV1 or mTRPV4), pcDNA5/FRT vector (containing polynucleotide encoding rTRPV1, rTRPV2, mTRPV3, rTRPV4, mTRPM8 or mTRPA1), and 600 ng/well of pCDNA3 (Invitrogen Corp., USA; containing green fluorescent protein (GFP) cDNA) using Fugene6 (Roche Diagnostics, USA) according to manufacturer's instruction. The transformed cells were cultured in DMEM/F12 medium containing 10% FBS and 1% penicillin/streptomycin in a CO₂ incubator for 24 hours. The cells were smeared on poly-L-lysine-coated glass coverslips, followed by further culture for 10-24 hours.

### EXAMPLE 2

### TRPV3 Activity Inhibition by TRPV3 Inhibitor

### <2-1> Treatment of Compounds

The TRPV3 transfected cell line prepared in Example 1 was treated with 10 μM camphor (Sigma-Aldrich, USA), during which 10 μM of isopentenyl pyrophosphate (Sigma-Aldrich, USA) was treated for a certain period of time. Stock solutions were made using water or DMSO, and were diluted with test solutions before use.

### <2-2> Measurement of Intracellular Calcium Level Changes by Calcium Imaging

Calcium imaging was performed with the transfected cell line treated by the method of Example <2-1>.

Particularly, the transfected cell line of Example <2-1> was loaded with Fluo-3AM (5 μM; Sigma Aldrich, USA) in the bath solution (140 mM NaCl, 5 mM KCl, 2 mM CaCl₂, 1 mM MgCl₂, 10 mM HEPES; adjusted to pH 7.4 with NaOH) containing 0.02% pluronic acid (Invitrogen, USA) at 37° C. for 1 hour. Calcium imaging was performed with LSM5 Pascal confocal microscope (Carl Zeiss, Germany), and time-lapse images (excitation 488 nm/emission 514 nm) were collected every 3 seconds using Carl Zeiss ratio tool software (Carl Zeiss, Germany). Mean value curve of calcium influx responses was made by Hill plot.

As a result, as shown in FIG. 1a, TRPV3 activity induced by camphor was inhibited by isopentenyl pyrophosphate.

### <2-3> Measurement of Intracellular Calcium Level Changes by Calcium Imaging

Calcium imaging was performed with the transfected cell line treated by the method of Example <2-1>.

Particularly, the transfected cell line of Example <2-1> was loaded with Fluo-3AM (5 μM; Sigma Aldrich, USA) in the bath solution (140 mM NaCl, 5 mM KCl, 2 mM CaCl₂, 1 mM MgCl₂, 10 mM HEPES; adjusted to pH 7.4 with NaOH) containing 0.02% pluronic acid (Invitrogen, USA) at 37° C. for 1 hour. Calcium imaging was performed with LSM5 Pascal confocal microscope (Carl Zeiss, Germany), and time-lapse images (excitation 488 nm/emission 514 nm) were collected every 3 seconds using Carl Zeiss ratio tool software (Carl Zeiss, Germany).

As a result, as shown in FIG. 1b, TRPV3 activity induced by camphor was inhibited by isopentenyl pyrophosphate, which was confirmed by Fluo-3 calcium imaging (n=59).

### EXAMPLE 3

### Investigation of Responses to TRPV3 Inhibitor in Different TRP Transfected Cell Lines

The TRPA1, TRPV1, TRPV2, TRPV3, and TRPM8 transfected cell lines prepared by the method of Example 1 and the non-transfected HEK cell line (control group) were treated with 10 μM of isopentenyl pyrophosphate. Calcium imaging was performed with the transfected cell lines treated as the above by the same manner as described in Example <2-2>.

As a result, as shown in FIG. 2, among the TRPs known to be expressed in sensory neurons and mediated pain, only TRPV3 was inhibited by isopentenyl pyrophosphate.

### EXAMPLE 4

### Inhibition of Cell Migration and Proliferation by TRPV3 Inhibitor

HaCat (ATCC, CCL-228) or HEK293T cells (ATCC CRL-11268) were seeded in a 24-well plate containing the medium containing camphor (4 mM). The present inventors drew lines 1 mm deep on the well fully filled with the cells to make artificial wound. The wounded cells were treated with IPP (10 μM), FPP (1 μM) and GPP (10 μM: geranyl pyrophosphate, Biomol, USA) respectively and incubated in a CO₂ incubator for 12 hours. The control group was not-treated. The width of recovered wound was measured under microscope and compared with that at the beginning, which was presented as wound recovery rate. All the experiments were performed on DMEM/FBS. Floating cells, which means dead cells, after making wound, were eliminated by using PBS to eliminate variables caused by dead cells. The cells were observed under microscope (×40), and distance was measured by using image analysis program (Meta-flour 7.1 Molecular Devices, USA).

As shown in FIG. 3, migration and proliferation of HaCat skin cells administered with 10 uM of isopentenyl pyrophosphate were investigated 12 hours after the treatment. As a result, migration and proliferation of the HaCat skin cells were inhibited by isopentenyl pyrophosphate and the inhibitory effect was most significant compared with those of other drugs. However, no significant inhibition was observed in HEK293 cells. The present inventors treated the cells with FPP alone and as a result, the inventors confirmed the inhibitory effect as well. But when FPP, GPP and IPP were treated with the medium containing 4 mM of the TRPV3 activator, camphor, inhibitory effect was not observed in HaCaT cells except the HaCaT cells treated with IPP. The above result suggests that camphor competes with FPP or there is synergy effect caused by FPP's another activity to a different target whether it is known or unknown. Consistent result was observed in HEK293 cell line that did not express TRPV3. However, IPP inhibits cell proliferation stronger than any other, far stronger than camphor. And no significant change was observed in the control HEK293 cell line that does not express TRPV3.

The Manufacturing Examples of the composition for the present invention are described hereinafter.

### MANUFACTURING EXAMPLE 1

### Preparation of Pharmaceutical Formulations

<1-1> Preparation of Powders

| | |
|---|---|
| Isopentenyl pyrophosphate | 2 g |
| Lactose | 1 g |

Powders were prepared by mixing all the above components, which were filled in airtight packs according to the conventional method for preparing powders.

<1-2> Preparation of Tablets

| | |
|---|---|
| Isopentenyl pyrophosphate | 100 mg |
| Corn starch | 100 mg |
| Lactose | 100 mg |
| Magnesium stearate | 2 mg |

Tablets were prepared by mixing all the above components by the conventional method for preparing tablets.

<1-3> Preparation of Capsules

| | |
|---|---|
| Isopentenyl pyrophosphate | 100 mg |
| Corn starch | 100 mg |
| Lactose | 100 mg |
| Magnesium stearate | 2 mg |

Capsules were prepared by mixing all the above components, which were filled in gelatin capsules according to the conventional method for preparing capsules.

<1-4> Preparation of Pills

| | |
|---|---|
| Isopentenyl pyrophosphate | 1 g |
| Lactose | 1.5 g |
| Glycerin | 1 g |
| Xylitol | 0.5 g |

Pills were prepared by mixing all the above components according to the conventional method for preparing pills. Each pill contained 4 g of the mixture.

<1-5> Preparation of Granules

| | |
|---|---|
| Isopentenyl pyrophosphate | 150 mg |
| Soybean extract | 50 mg |
| Glucose | 200 mg |
| Starch | 600 mg |

All the above components were mixed, to which 100 mg of 30% ethanol was added. The mixture was dried at 60° C. and the prepared granules were filled in packs.

### MANUFACTURING EXAMPLE 2

### Preparation of Cosmetic Composition

<2-1> Preparation of Skin Lotion

Skin lotion containing isopentenyl pyrophosphate of the present invention as an active ingredient was prepared according to the composition shown in Table 1.

TABLE 1

| | Content |
|---|---|
| Raw material | (weight part) |
| Isopentenyl pyrophosphate | 10.0 |
| 1,3-butyleneglycol | 1.00 |
| Disodium EDTA | 0.05 |
| Allantoin | 0.10 |
| Dipotassium glycyrrhizinate | 0.05 |
| Citric acid | 0.01 |
| Sodium citrate | 0.02 |
| Glycereth-26 | 1.00 |
| Arbutin | 2.00 |
| Hydrogenated castor oil | 1.00 |
| Ethanol | 30.00 |
| Preservative | Small amount |
| Colorant | Small amount |
| Flavor | Small amount |
| Purified water | Small amount |

<2-1> Preparation of Nutrition Cream

Nutrition cream containing isopentenyl pyrophosphate of the present invention as an active ingredient was prepared according to the composition shown in Table 2.

TABLE 2

| Raw material | Content (weight part) |
|---|---|
| Isopentenyl pyrophosphate | 10.0 |
| 1,3-butyleneglycol | 7.0 |
| Glycerin | 1.0 |
| D-panthenol | 0.1 |
| Plant extract | 3.2 |
| Magnesium aluminum silicate | 0.3 |
| PEG-40 stearate | 1.2 |
| Stearic acid | 2.0 |
| Polysorvate 60 | 1.5 |
| Lipophilic glyceryl stearate | 2.0 |
| Sorbitan sesquioleate | 1.5 |
| Cetearyl alcohol | 3.0 |
| Mineral oil | 4.0 |
| Squalane | 3.8 |
| Caprylic/Capric triglyceride | 2.8 |
| Vegetable oil | 1.8 |
| Dimethicone | 0.4 |
| Dipotassium glycyrrhizinate | Small amount |
| Allantoin | Small amount |
| Sodium hyaluronate | Small amount |
| Tocopheryl acetate | Proper amount |
| Triethanolamine | Proper amount |
| Preservative | Proper amount |
| colorant | Proper amount |
| Purified water | Proper amount |

### MANUFACTURING EXAMPLE 3

### Preparation of Dairy Products

5˜10 weight part of isopentenyl pyrophosphate of the present invention was added to milk. Health enhancing dairy products such as butter and ice cream were prepared with the milk mixture according to the conventional method.

### MANUFACTURING EXAMPLE 4

### Preparation of Beverages

| | | |
|---|---|---|
| Isopentenyl pyrophosphate | 1000 | mg |
| Citric acid | 1000 | mg |
| Oligosaccharide | 100 | g |
| Maesil (Prunus mume) Extract | 2 | g |
| Taurine | 1 | g |
| Purified water | up to 900 | ml |

The above constituents were mixed according to the conventional method for preparing health beverages. The mixture was heated at 85° C. for 1 hour with stirring and then filtered. The filtrate was loaded in 2 liter sterilized containers, which were sealed and sterilized again, stored in a refrigerator until they would be used for the preparation of a composition for health beverages. The constituents appropriate for favorite beverages were mixed according to the preferred mixing ratio but the composition ratio can be adjusted according to regional and national preferences, etc.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for treating skin disease containing the step of administering a pharmaceutically effective dose of isopentenyl pyrophosphate to a subject with skin disease or applying the same on the skin of the subject.

2. The method for treating skin disease according to claim 1, wherein the skin disease is caused by wound healing process and over-proliferation of cells.

3. The method for treating skin disease according to claim 2, wherein the skin disease is selected from the group consisting of psoriasis, lichen planus, keratosis, basal cell carcinoma, hypersensitive dermatitis, atopic dermatitis, seborrheic dermatitis, and keloid.

4. The method for treating skin disease according to claim 1, wherein the isopentenyl pyrophosphate is administered to a subject in the form of a pharmaceutical composition.

5. The method for treating skin disease according to claim 1, wherein the isopentenyl pyrophosphate is applied on the skin in the form of a cosmetic composition.
